(19) United States
(12) Patent Application Publication
(10) Pub. No.: US 2006/0224172 A1
(43) Pub. Date: Oct. 5, 2006

(54) Blood sampling device

(75) Inventors: Richard W. LeVaughn, Newman, GA (US); Gwenn E. Kennedy, Ellenwood, GA (US); Stephen J. Flynn, Peachtree City, GA (US); Carl E. Griffin, Marietta, GA (US); John C. Irwin, Woodstock, GA (US); Mary Kate Pynes, Dallas, GA (US); Stephanie J. Campbell, Kennesaw, GA (US); Christopher J. Ruf, Marietta, GA (US); Mitchell A. Solis, Cumming, GA (US); Avi M. Robbins, Longwood, FL (US); Jason R. Heath, Marietta, GA (US)

Correspondence Address: GARDNER GROFF SANTOS & GREENWALD, P.C., 2018 POWERS FERRY ROAD, SUITE 800, ATLANTA, GA, 30339, US

(73) Assignee: Facet Technologies, LLC, Marietta, GA (US)

(21) Appl. No.: 10/569,023

(22) PCT Filed: Aug. 19, 2004
(86) PCT No.: PCT/US04/26972

§ 371 Date: Feb. 17, 2006

Related U.S. Application Data
(60) Provisional application No. 60/497,025, filed on Aug. 20, 2003.

(51) Int. Cl. A61B 17/32

(52) U.S. Cl. 606/181

(57) ABSTRACT

A sampling device having a lancing apparatus, and optionally a detector and a meter. The lancing apparatus includes a replaceable cartridge of lancets, each lancet having an independent arming and drive mechanism enabling non-sequential and/or simultaneous firing of lancets. A lancet retainer independently associated with each lancet is movable to uncover an opening through which the lancet tip passes, and to release the lancet for activation. The lancets can be actuated in any order, and multiple lancets can be simultaneously actuated, if desired.

### CROSS-REFERENCE TO RELATED APPLICATIONS

This application claims the benefit of U.S. Provisional Patent Application Ser. No. 60/497,025, filed Aug. 20, 2003, the entire content of which is hereby incorporated herein by reference for all purposes.

### TECHNICAL FIELD

The present invention relates generally to medical devices and procedures, and more particularly to lancing devices for the collection and/or analysis of samples of blood or other bodily fluid.

### BACKGROUND OF THE INVENTION

Many medical procedures require puncturing of the skin, and sometimes underlying tissues, of an animal or human subject. For example, a sharp lancet tip is commonly used to puncture the subject's skin at a lancing site to obtain a sample of blood, interstitial fluid or other body fluid, as for example in blood glucose monitoring by diabetics, and in blood typing and screening applications. In some instances, a person must periodically sample their blood for multiple testing throughout the day or week. Because re-use of a lancet can result in infection or spread of bloodborne contaminants, persons requiring repeated testing often must carry multiple lancets with them. This can be inconvenient and lead to reduced compliance with a prescribed test regimen. Accordingly, it can be seen that needs exist for a convenient, compact multi-use lancing device.

Various devices are known for sampling blood and other body fluids for analysis of the condition of a human or other animal subject. For example, U.S. Pat. No. 5,971,941 is understood to show a cassette with test strips for placement by a slider. A lancet pierces the skin surface so that blood can be obtained for analysis. The lancets are integrated on a test strip, and are positioned together with the test strip. Another embodiment is understood to show a disposable cylindrical insert having a lancet and a test membrane with an aperture for the lancet. The insert is inserted in a mounting cavity of a plunger or piston, which forces the lancet outward for blood withdrawal. DE 198 19 407 A1 is understood to show a multiplicity of test strips with integrated lancets for insertion into an analysis device.

U.S. Pat. No. 4,787,398 is understood to show a device with a plunger for directing a lancet outward, and has an evaluation system and a display system. A replaceable unit is applied to the device for each measurement. The replaceable unit comprises the lancet and a test strip, which is wetted with blood. This replaceable unit is thrown away after each use. EP 0 449 525 A1 is understood to show a blood withdrawal system wherein a new lancet is inserted manually into a release device before each use. A test strip is then inserted into the device. U.S. Pat. No. 4,627,445 is understood to show a device for measuring blood sugar, with an integrated blood withdrawal unit. A new replaceable lancet and test elements must be installed to the device for testing, and afterward disassembled. U.S. Pat. No. 5,951,492 is understood to show a disposable unit with a capillary tube and a test strip, to which sampled blood taken is applied. The capillary tube includes a lancet. A new disposable unit is attached and removed before and after each measurement.

EP 0877250 A2, EP 0949506 A2 and EP 811843 A2 are understood to show devices having a multiplicity of test elements arranged on a rotatable disk carrier. The test elements are brought successively into a working position and pushed out of the housing to be wetted with blood. U.S. Pat. No. 6,228,100 and U.S. Pat. No. 4,794,926 are understood to show lancets arranged on a carrier, which is rotated with respect to a housing.

German Application DE 100 57 832 C1 is understood to show a lancing device of a known form. Other lancing devices understood to include multiple lancets are shown, for example, in U.S. Pat. No. 6,540,675 and WO 02/36010 A1. EP 0589186 B1 is understood to show a lancet with a removable protective cap. WO 01/66010 A1 is understood to include a multiplicity of lancets in a magazine, with an opening of the chamber closed by an elastic material, which is penetrated in the puncture process.

Known sampling devices have, however, not proven fully satisfactory to all users for a variety of reasons. Accordingly, it can be seen that needs exist for a convenient, compact multi-use lancing device. Needs also exist for a compact sampling device that includes lancets, test media and a sample analysis device in a combined apparatus. It is to the provision of an improved sampling device meeting these and other needs that the present invention is primarily directed.

### SUMMARY OF THE INVENTION

Briefly described, example embodiments of the present invention include an improved sampling device that is convenient, compact, and includes multiple lancets in a single cassette or cartridge. Further example embodiments of the present invention include a compact sampling device that includes lancets, and optionally further includes test media and a sample analysis device in a combined apparatus. The present invention preferably increases convenience for the user, thereby encouraging more frequent testing and insuring compliance with the subject's prescribed testing regimen.

In one aspect, the invention is a lancing device including a plurality of lancets. Each lancet has a separate drive spring independently associated therewith to propel the lancet along a lancing stroke from a retracted position to an extended position.

In another aspect, the invention is a lancing device having at least one lancet with a detachable endcap, and a lancet retainer independently associated with each lancet. The lancet retainer is movable between a first position and a second position, and movement of the lancet retainer between the first position and the second position removes the detachable endcap from the lancet and retracts the lancet into a cocked position.

In still another aspect, the present invention is a lancing device including a plurality of lancets, wherein the lancets can be fired in any order.

In another aspect, the invention is a lancing device including a carrier base defining a plurality of guide channels; and a cover mounted on the carrier base to define a housing enclosing the plurality of guide channels and defining a plurality of openings, each opening aligned with an end of one of the guide channels. The lancing device preferably also includes a plurality of lancets, each lancet having a body with a sharp tip extending therefrom and an endcap removably positioned over the sharp tip, each lancet associated with one of the guide channels and traversing a path between a retracted position fully within the housing and an extended position wherein at least the sharp tip projects through one of the openings. A plurality of drive springs are preferably also included, each spring coupled at a first end to the housing and at a second end to the body of one of the lancets. The device preferably also includes a plurality of lancet retainers, each lancet retainer associated with one of the openings in the housing, and movable between a first position covering that opening and a second position uncovering that opening, and wherein each lancet retainer retains its associated lancet in the first position and releases the associated lancet in the second position. A trigger is preferably included, and is operable to actuate any of the plurality of lancets that have been released by its associated lancet retainer.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of an assembled sampling device according to an example embodiment of the present invention.

FIG. 2 is an exploded view of the sampling device of FIG. 1, showing meter, detector, and lancet cartridge components of the apparatus separated from one another.

FIG. 3 is a perspective view of a lancet cartridge portion of the sampling device of FIG. 1, with a cover portion removed to show inner components, according to an example embodiment of the present invention.

FIG. 4 is an exploded view of a lancet cartridge of the sampling device of FIG. 1, according to an example embodiment of the present invention.

FIG. 5A is a side view of the assembled lancet cartridge of the sampling device of FIG. 1, according to an example embodiment of the present invention.

FIG. 5B is a cross sectional top view of the lancet cartridge taken along axis A-A of FIG. 5A.

FIG. 6 is another perspective view of a lancet cartridge according to an example embodiment of the present invention, with the cover portion removed.

FIG. 7 is a perspective view of the inner face of a cover portion of a lancet cartridge according to an example embodiment of the present invention.

FIG. 8 is a perspective view of a meter portion of a sampling device according to an example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

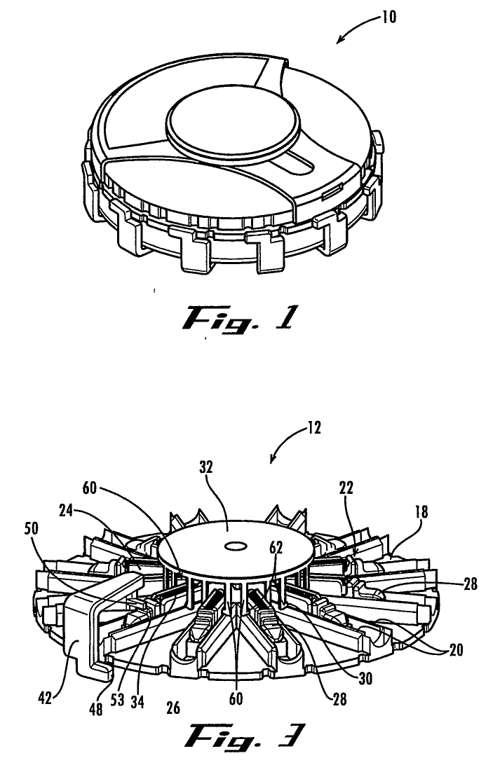

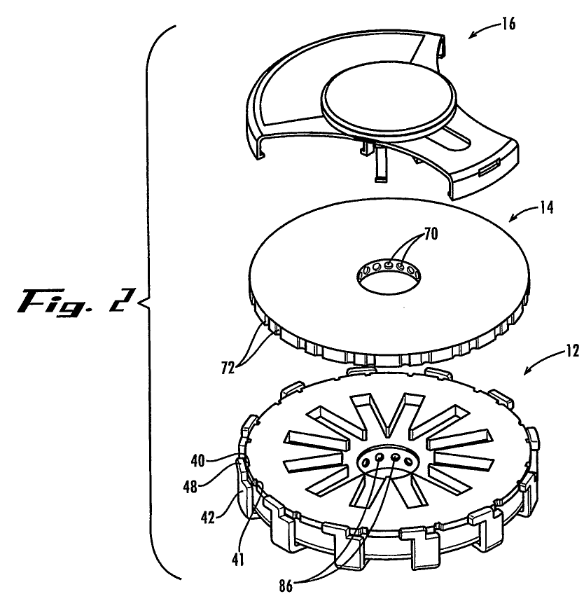

With reference now to the drawing figures, a blood sampling device 10 is shown byway of example embodiments of the present invention. As seen in FIGS. 1 and 2, in one embodiment of the invention, the blood sampling device 10 comprises an apparatus combining a lancet cartridge component 12 containing one or more lancets for piercing the skin to generate a sample of blood or other body fluid, a detector component 14 containing test strips or other media for sample collection and/or processing, and a meter component 16 for analyzing a constituent analyte of a collected sample. In alternate embodiments, the invention comprises any one of these components separately, or any two or more components in combination.

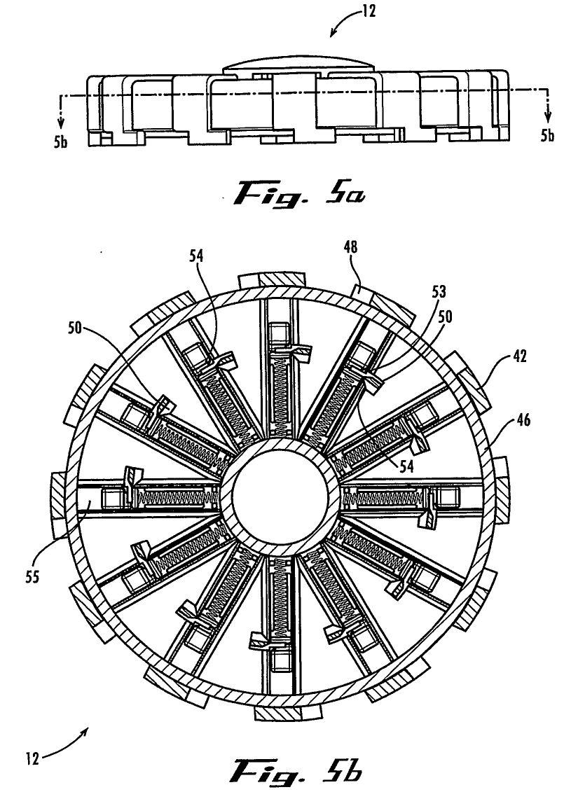

FIGS. 3-7 show further details of the lancet cartridge 12 of the present invention. The lancet cartridge 12 preferably comprises a disk-shaped lancet carrier or base 18 that optionally can be rotationally coupled to one or more other components such as meter 16 and/or detector 14 in a combination apparatus, or can separately comprise a standalone multi-use lancing device. As seen best in FIG. 3, in an example embodiment, the base 18 is a generally circular disk having a circular central opening, and preferably includes a plurality of guide channels, defined between adjacent parallel pairs of radially extending lancet tracks 20, having lancets 22 slidably disposed therebetween.

Each lancet 22 preferably comprises a body 24, having a needle or blade (not shown) mounted therein, with a sharp tip projecting from one end of the body and covered with an end cap 28 to maintain sterility and protect against accidental contact or needle-sticks. The lancet body 24 preferably further defines at least one recess 30 for engagement with a triggering mechanism 32.

In the example embodiments, a spring 34 is provided to drive the lancet from a retracted position wherein the sharp tip is shielded within the cartridge and an extended position wherein the sharp tip extends a distance outwardly through an opening of the cartridge. The spring 34 optionally also serves to retract the lancet tip back within the cartridge after firing, or alternatively a separate return spring is provided. In further alternate embodiments, a cam, motor or other drive means propels the lancet from its retracted position to its extended position. In example embodiments, each lancet 22 is associated with its own spring 34, such that each lancet has its own independent drive mechanism. In this manner, the lancets can be delivered to the end user in an initially energized or “cocked” configuration, with each drive spring compressed and ready for firing, thereby improving user convenience by eliminating the need to cock the device before each lancing operation. In alternative embodiments, the device is delivered with the springs relaxed and each lancet 22 is initially positioned in an “uncocked” state such that the device is cocked by the user before lancing.

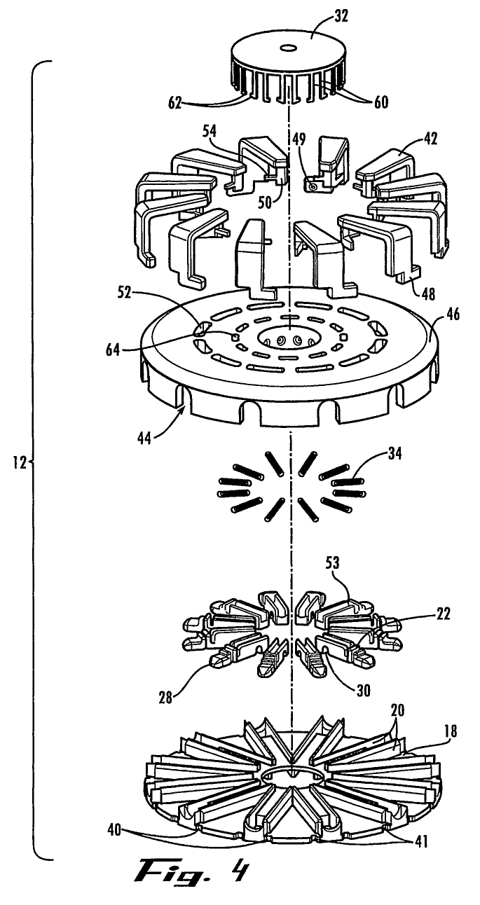

Each lancet 22 is preferably translationally mounted between a pair of lancet tracks 20, which define a guide channel or path of travel between the retracted and extended positions. A lancet retainer 42 retains each lancet 22 in a first position along the path of travel until the lancet is to be made ready for actuation or firing to lance the skin at a sampling site. As noted above, the first position may be a cocked position or an uncocked position. As seen in the example embodiment of FIGS. 4, 5 and 6, the lancet retainers 42 each comprise an outer flange that covers a respective one of a plurality of openings 44 through the rim of a top plate or cover 46 of the cartridge. Each lancet retainer 42 also includes an inner arm 50 that projects through a respective one of a plurality of slotted openings 52 through the cover 46 and into engagement with a cooperating recess 53 of a respective lancet 22 to prevent firing of that lancet upon activation of a triggering mechanism 32 unless that lancet has been made ready for firing. Each lancet retainer 42 preferably further comprises a resilient tab 48 projecting from its outer flange with a projection 49 thereon that alternatively engages a first recess 40 formed in the outer rim of the base 10 to secure the lancet retainer in a first position covering its respective opening 44, or a second recess 41 formed in the outer rim of the base to secure the lancet retainer in a second position exposing its respective opening 44.

A specified lancet 22 is made ready for firing by moving the lancet retainer 42 associated with that lancet from its first position to its second position. This simultaneously exposes the opening 44 in the housing through which the sharp tip of the lancet projects in its extended position, and moves the inner arm 50 out of engagement with the cooperating recess 53 of the lancet to free the lancet to slide along the path of travel of the lancing stroke. The arm 50 preferably further comprises an inclined face forming a wedge 54 that separates the protective endcap 28 from the lancet being made ready for firing. As the lancet retainer 42 is moved from its first position to its second position, the wedge 54 is driven through the recess 53, causing detachment of the endcap 28 from the rest of the lancet 22. Alternatively or additionally, the endcap 28 comprises an inclined face for interacting with a flat or otherwise configured portion of the wedge 54. The detached end cap is preferably then deposited into a recess or well 55, beneath or otherwise out of the way of the lancet's path of travel. The lancet cap may be moved into the recess 55 by a spring member, gravity, magnetic attraction, or other biasing means, and optionally is replaced onto the lancet tip after firing. For example, in one embodiment of the invention, the endcap 28 comprises a magnetic material and a magnet is disposed in the housing adjacent the recess 55 to move the detached endcap into the recess. One or more fins or other guide members are optionally formed in the housing to guide the detached endcap 28 into the recess 55. If the lancets 22 are not initially provided in a cocked position, movement of the lancet retainer 42 from its first position to its second position can also serve to cock the lancet. As the wedge 54 moves across a cooperating surface of the lancet, the inclined face of the wedge acts as a cam to drive the lancet radially inwardly in the direction of the lancet's retracted position. Wedge 54 can be configured to simultaneously or sequentially decap the lancet and cock the lancing device. For example, by controlling the placement of the contact faces along either or both sides of the wedge 54, the endcap can be removed prior to, simultaneously with, or after the lancet is retracted to cock the device.

Because each lancet 22 preferably includes its own associated drive means, independent of the other lancets, and because each lancet retainer 42 is operable independent of the other lancet retainers, the lancets 22 can be used in any order as determined by the user. After firing a lancet, the associated lancet retainer 42 is preferably returned to its first position to the cover the opening 44 to prevent accidental contact with a contaminated lancet tip and to prevent foreign debris from entering the device.

The lancing apparatus of the present invention preferably further comprises a triggering mechanism for releasing one or more lancets 22 that have been made ready for firing as described above. Upon releasing the triggering mechanism, the lancet 22 is driven from its cocked or retracted position, along the path of travel of the lancing stroke under the influence of the drive means, into the extended position for lancing the skin of a subject placed against or adjacent the opening 44. The triggering mechanism preferably comprises a trigger button 32 having one or more cantilevered resilient fingers 60 extending therefrom for engaging each lancet 22. With reference to FIGS. 3, 4 and 6, in the depicted embodiment, a plurality of pairs of fingers 60 are provided, each pair engaging a respective one of the lancets 22. Each of the fingers 60 preferably comprises a barb 62 at its free end, for releasable engagement with a cooperating recess 30 in the side of the body 24 of the associated lancet 22. Slots or openings 64 are preferably provided through the top cover plate 46 for passage of the fingers 60.

Prior to releasing the triggering mechanism to fire a lancet 22, the barbs 62 engage the recesses 30 to hold the lancet in the retracted or cocked position. When the triggering mechanism is released, interaction of an inclined face of the barb 62 with a cooperating surface of the lancet tracks 20 spreads the pair of fingers 60 apart and out of engagement with the recesses 30, releasing any lancet 22 that has been made ready for firing as described above. Lancets that have not been made ready for firing remain constrained in their retracted positions by their associated lancet retainers 42, and are not fired upon releasing the triggering mechanism. In example embodiments, two or more lancets can be made ready for firing, and can be fired simultaneously to lance the skin at multiple lancing sites, so that multiple samples can be separately analyzed or a larger volume of body fluid collected. This can help reduce discomfort or anxiety that might otherwise result if multiple lancing operations were performed sequentially rather than simultaneously. Alternatively, interlocks can be provided to prevent more than one lancet from being fired at once. The device optionally comprises means for preventing re-use of a lancet after it has been fired, such as a one-way latching mechanism or a locking element that blocks the lancet path after a single use to prevent re-cocking or re-firing.

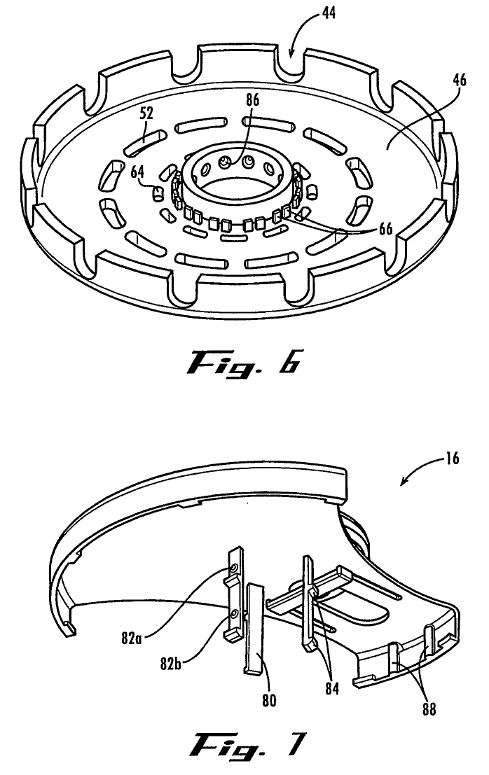

FIG. 7 shows the inside of the top plate or cover 46 of the lancing device according to an example embodiment of the invention. One or more posts 66 preferably project from the cover 46 for attachment of an inner end of the drive spring 34. In the depicted embodiment, a plurality of pairs of posts 66 are provided, each pair configured to hold a drive spring 34 captive therebetween. The outer end of the spring is preferably securely attached to the lancet 22, for example by capture between two posts 26 projecting from the lancet body. By attaching the inner end of the spring 34 to the housing 46 and the outer end of the spring to the lancet 22, the spring can function both as a drive spring to propel the lancet from the retracted position to the extended position upon firing, and as a return spring to retract the lancet back into the housing after lancing. In alternate embodiments, either or both ends of the spring remain unattached from the lancet and/or the housing. One or more recesses or other surface features 86 are preferably formed in the inner hub surface of the central opening in the cover 46 for engagement with a cooperating element of the detector 14 and/or meter 16, as described further below.

As seen in FIGS. 1 and 2, an example embodiment of the detector component 14 of the sampling device comprises a generally circular disk having a circular central opening. The detector 14 is preferably shaped to generally match the profile of the lancing device component 12 and the meter 16, and is configured for detachable and rotatable coupling with the lancing apparatus 12 and the meter 16. The detector 14 preferably comprises a plurality of test strips, assay material or other test media for collecting and/or processing a sample of blood or other body fluid to detect and/or quantify a specified analyte such as blood glucose, cholesterol, blood cell counts, gases such as oxygen and carbon dioxide, or salts such as potassium and sodium. The detector 14 may further comprise a chemical detection system, such as for example, an enzyme linked immunosorbant assay (ELISA), an optic detection system, or combinations thereof, such as for example, the use of immunofluorescence (IFA).

The detector 14 can comprise the same or a different number of test media elements as the lancing apparatus 12 has lancets. In example embodiments, the detector 14 is independently rotatable from the lancing apparatus 12 and the meter 16. In alternate embodiments, the detector's 14 rotation is indexed to the rotation of the base disc 18 of the lancing apparatus. The detector 14 preferably has one or more recesses or other surface features 70 formed in an inner hub surface of its central opening for interaction with cooperating elements of the lancing apparatus 12 and/or the meter 16. One or more indentations, ridges, or other surface features 72 are preferably located along the outer rim of the detector 14 for alignment and indexing of the test media with the meter 16.

As seen best in FIGS. 1, 2 and 8, an example embodiment of the meter 16 preferably comprises a body portion having one or more legs 80 projecting therefrom for engaging the lancing apparatus 12 and/or the detector 14. Shoulders 84 are preferably provided on the legs 80 for providing a snap-fit connection with the lancing apparatus 12 and/or the detector 14, to retain the apparatus in an assembled configuration. One or more protrusions or other surface features 82 on the legs interact with the recesses 70, 86 of the detector 14 and/or the lancing apparatus 12 for alignment and indexing of relative rotation between the components, to provide an audible click or tactile indexing as the detector and/or the lancing apparatus are rotationally advanced. Alternatively or additionally, one or more ridges or other surface features 88 are preferably provided on an inner rim surface of the meter 16 for interaction with the indentations 72 or other surface features of the detector 14 to provide indexed rotation and alignment between these components. The meter 16 preferably comprises electronic processing means of standard varieties known in the art for analyzing and/or displaying sample test results.

The device is assembled for use by snapping the detector 14 in place with the legs 80 of the meter 16 extending through the central opening of the detector and the proximal shoulders 84 snap-fit over the detector. The lancing apparatus 12 is then installed over the detector with the legs 80 extending through its central opening and the distal shoulders 84 snap-fit over the lancing apparatus. The lancing apparatus 12 and the detector 14 are preferably symmetric, so that no alignment of these components with the meter is required upon assembly. And because the lancets 22 can be fired independently and in any order, it is not necessary to load the lancet cartridge of the present invention in any specified orientation. One or more of the lancet retainers 42 of the lancing apparatus 12 are moved circumferentially along the rim of the lancing apparatus, from the first position to the second position, to expose an associated opening 44 and to make an associated lancet ready for firing as described in greater detail above. The user then places the skin of a desired sampling site against or adjacent the exposed opening 44. The device is triggered by pressing trigger button 32, and the sharp tip of the lancet is driven through its lancing stroke, from the retracted position to the extended position where it pierces the skin of the sampling site. A sample of blood is expressed from the wound at the sampling site and optionally is transferred to test media of the detector 14. The sample is processed and information regarding an analyte is displayed on the meter 16. After lancing, the lancet retainer 42 is returned to its first position to re-cover the opening 44, and the device is safe for storage or ready for subsequent use. One or both of the lancing apparatus 12 and/or the detector 14 can be removed, disposed of, and replaced onto the reusable meter 16 when fully used. Optionally, interchangeable replacement lancet cartridges can be provided with lancets having tips of different standard lengths, whereby a user can select a desired depth of penetration by appropriate selection of the lancet cartridge to be installed.

While the invention has been described with reference to preferred and example embodiments, it will be understood by those skilled in the art that a variety of modifications, additions and deletions are within the scope of the invention, as defined by the following claims.

What is claimed is:

1. A lancing device comprising a plurality of lancets, wherein each lancet has a separate drive spring independently associated therewith to propel the lancet along a lancing stroke from a retracted position to an extended position.

2. The lancing device of claim 1, further comprising a lancet retainer associated with each lancet, the lancet retainer movable between a first position in engagement with the lancet for holding the lancet in the retracted position, and a second position out of engagement with the lancet.

3. The lancing device of claim 2, further comprising a housing defining a plurality of openings, each opening associated with a respective one of said plurality of lancets, and wherein each said lancet retainer covers a respective one of said plurality of openings when the lancet retainer is in its first position.

4. The lancing device of claim 2, wherein each said lancet retainer comprises an arm having an inclined face for engagement with a cooperating surface of the associated lancet as the lancet retainer moves from its first position to its second position.

5. The lancing device of claim 4, wherein the inclined face interacts with a cooperating portion of an endcap of the lancet to detach said endcap from the lancet.

6. The lancing device of claim 4, wherein the inclined face interacts with the lancet to move the lancet into a cocked position.

7. The lancing device of claim 1, wherein the plurality of lancets are arranged in a plane, and wherein each lancet comprises a detachable endcap that is moved out of said plane upon detachment.

8. The lancing device of claim 1, further comprising a base having a plurality of pairs of lancet tracks, each pair of lancet tracks defining a path of travel of the lancing stroke for an associated one of the plurality of lancets.

9. The lancing device of claim 1, further comprising a trigger button having a plurality of resilient fingers extending therefrom, each of said fingers releasably engaging one of said plurality of lancets.

10. The lancing device of claim 9, wherein actuation of said trigger button simultaneously releases each of said fingers from its associated lancet.

11. A sampling device comprising the lancing device of claim 1, releasably coupled to a detector component comprising test media, and to a meter for analyzing a collected sample of body fluid.

12. A lancing device comprising:
at least one lancet having a detachable endcap; and
a lancet retainer independently associated with each said at least one lancet, the lancet retainer movable between a first position and a second position,
wherein movement of the lancet retainer between the first position and the second position removes the detachable endcap from the lancet and retracts the lancet into a cocked position.

13. The lancing device of claim 12, further comprising a housing defining an opening associated with each said at least one lancet, and wherein the lancet retainer associated with said lancet covers the opening in the first position and exposes the opening in the second position.

14. The lancing device of claim 12, further comprising a separate drive mechanism for each lancet.

15. The lancing device of claim 12, wherein the lancet retainer comprises a wedge, and wherein upon movement of the lancet retainer between the first position and the second position, said wedge drives the detachable endcap in an outward direction and drives the lancet in an inward direction.

16. The lancing device of claim 12, comprising a plurality of lancets and a trigger capable of simultaneously releasing more than one lancet.

17. A lancing device comprising a plurality of lancets, wherein the lancets can be fired in any order.

18. The lancing device of claim 17, wherein each lancet comprises a separate and independently operable drive mechanism.

19. The lancing device of claim 18, wherein lancet is delivered to the user with the drive mechanism of each lancet cocked for firing.

20. The lancing device of claim 19, further comprising a plurality of lancet retainers, each lancet retainer associated with one of said plurality of lancets and movable between a first position in engagement with the associated lancet and a second position out of engagement with the associated lancet.

21. A lancing device comprising:
a carrier base defining a plurality of guide channels;
a cover mounted on the carrier base to define a housing enclosing the plurality of guide channels and defining a plurality of openings, each opening aligned with an end of one of the guide channels;
a plurality of lancets, each lancet having a body with a sharp tip extending therefrom and an endcap removably positioned over the sharp tip, each lancet associated with one of the guide channels and traversing a path between a retracted position fully within the housing and an extended position wherein at least the sharp tip projects through one of the openings;
a plurality of drive springs, each spring interfacing at a first end with the housing and interfacing at a second end with one of the lancets;
a plurality of lancet retainers, each lancet retainer associated with one of the openings in the housing, and movable between a first position covering that opening and a second position uncovering that opening, and wherein each lancet retainer retains its associated lancet in the first position and releases the associated lancet in the second position; and
a trigger operable to actuate any of the plurality of lancets released by its associated lancet retainer.

22. The lancing device of claim 21, wherein each lancet retainer comprises a wedge, and wherein movement of the lancet retainer from the first position to the second position drives the wedge between the body and the endcap of the associated lancet to remove the endcap and uncover the sharp tip.

23. The lancing device of claim 22, wherein each guide channel comprises a well for receiving the endcap of the associated lancet after separation from the lancet body, and retaining the endcap out of the path of the lancet.

24. The lancing device of claim 22, wherein movement of the lancet retainer from the first position to the second position also energizes the associated drive spring.
